(19) United States
(12) Patent Application Publication
(10) Pub. No.: US 2010/0305672 A1
(43) Pub. Date: Dec. 2, 2010

(54) TIP ASSEMBLY FOR MEDICAL ELECTRICAL LEAD

(76) Inventors: Michael A. Felling, Osceola, WI (US); Daniel J. Foster, Lino Lakes, MN (US); Joshua F. Adamietz, Hugo, MN (US); Kimberly A. Jorgensen, Minneapolis, MN (US); Duane T. Meyer, St. Paul, MN (US)

Correspondence Address: FAEGRE & BENSON LLP;PATENT DOCKETING - INTELLECTUAL PROPERTY (32469), 2200 WELLS FARGO CENTER, 90 SOUTH SEVENTH STREET, MINNEAPOLIS, MN, 55402-3901, US

(21) Appl. No.: 12/785,941

(22) Filed: May 24, 2010

Related U.S. Application Data
(60) Provisional application No. 61/181,954, filed on May 28, 2009.

(51) Int. Cl. A61N 1/05

(52) U.S. Cl. 607/116

(57) ABSTRACT

An implantable lead may include a coupler, a fixation helix secured to the coupler and a guide element that includes an engaging surface and a proximal bearing surface. The engaging surface may be configured to engage the fixation helix such that the engaging surface of the guide element interacts with the fixation helix to cause the fixation helix to translate longitudinally when the fixation helix is rotated against the engaging surface. Longitudinal translation of the coupler and fixation helix may be limited by the distal end of the coupler contacting the proximal bearing surface of the guide element.

### CROSS-REFERENCE TO RELATED APPLICATIONS

This application claims benefit of U.S. Provisional Application No. 61/181,954, filed on May 28, 2009, entitled “Tip assembly for Medical Electrical Lead,” which is incorporated herein by reference in its entirety.

### TECHNICAL FIELD

The present invention relates to implantable medical devices and relates more particularly to leads for cardiac rhythm management (CRM) systems.

### BACKGROUND

Various types of medical electrical leads for use in cardiac rhythm management (CRM) and neurostimulation systems are known. For CRM systems, such leads are typically extended intravascularly to an implantation location within or on a patient's heart, and thereafter coupled to a pulse generator or other implantable device for sensing cardiac electrical activity, delivering therapeutic stimuli, and the like. The leads frequently include features to facilitate securing the lead to heart tissue to maintain the lead at its desired implantation site.

### SUMMARY

The present invention, according to one embodiment, is an implantable lead that has a flexible body, a connector assembly that is secured to a proximal end of the body for coupling the lead to an implantable medical device, a conductor member disposed longitudinally within the body and a distal assembly coupled to a distal end of the body. The connector assembly includes a terminal pin that is rotatable relative to the body. The conductor member is coupled to the terminal pin and is rotatable relative to the body. The distal assembly includes a housing having a distal region and a proximal region, the proximal region fixedly coupled to the distal end of the body. A coupler is rotatably disposed within the housing, the coupler having a proximal end and a distal end, the proximal end connected to the conductor member, a helical electrode fixedly secured to the coupler, and a guide element connected to or integral with the housing located distal to the coupler. The guide element includes an engaging surface and a proximal bearing surface, the engaging surface configured to engage the helical electrode. The engaging surface of the guide element interacts with the helical electrode to cause the helical electrode and the coupler to translate longitudinally when the helical electrode is rotated against the engaging surface, and longitudinal translation is limited by the distal end of the coupler contacting the proximal bearing surface of the guide element.

The present invention, according to another embodiment, is an implantable lead that is configured to carry an electrical signal. The implantable lead includes a flexible body that extends between a proximal end and a distal end and that is configured to carry an electrical signal from the proximal end to the distal end, and a distal assembly coupled to the distal end of the body. The distal assembly includes a housing having a distal region and a proximal region, the proximal region fixedly coupled to the distal end of the body, the distal region including a distal end. A coupler is rotatably disposed within the housing, the coupler having a proximal end and a distal end, the proximal end connected to the conductor member, a fixation helix fixedly secured to the coupler, and a guide element connected to or integral with the housing located distal to the coupler. The guide element includes an engaging surface and a proximal bearing surface, the engaging surface configured to engage the helical electrode. The engaging surface of the guide element interacts with the fixation helix to cause the fixation helix and the coupler to translate longitudinally when the fixation helix is rotated against the engaging surface, and longitudinal translation is limited by the distal end of the coupler contacting the proximal bearing surface of the guide element.

The present invention, according to another embodiment, is an implantable lead having a flexible body extending between a proximal end and a distal end, a connector assembly secured to the proximal end for coupling the lead to an implantable medical device, the connector assembly including a terminal pin rotatable relative to the body, a conductor member disposed longitudinally within the body and coupled to the terminal pin, the conductor member rotatable relative to the body, and a distal assembly that is coupled to the distal end of the body. The distal assembly includes a housing having a distal region, a distal end and a proximal region, the proximal region fixedly coupled to the distal end of the body. A coupler is rotatably disposed within the housing, the coupler having a proximal end and a distal end, the proximal end connected to the conductor member, a helical electrode fixedly secured to the coupler. A distal plate that is connected to or integral with the housing is located within the distal region of the housing, the distal plate including a proximal bearing surface and an arcuate opening defining an engaging surface, the engaging surface configured to engage the helical electrode. The engaging surface interacts with the helical electrode to cause the helical electrode and the coupler to translate longitudinally when the helical electrode is rotated against the engaging surface, and longitudinal translation is limited by the distal end of the coupler contacting the proximal bearing surface of the distal plate.

The present invention, according to another embodiment, is an implantable lead that has a flexible body, a connector assembly that is secured to a proximal end of the body for coupling the lead to an implantable medical device, a conductor member disposed longitudinally within the body and a distal assembly coupled to a distal end of the body. The connector assembly includes a terminal pin that is rotatable relative to the body. The conductor member is coupled to the terminal pin and is rotatable relative to the body. The distal assembly includes a housing having a distal region and a proximal region, the proximal region fixedly coupled to the distal end of the body. A coupler is rotatably disposed within the housing, the coupler having a proximal end and a distal end, the proximal end connected to the conductor member, a helical electrode fixedly secured to the coupler, and a guide element insert disposed within the distal region of the housing. The guide element insert includes an engaging surface and a proximal bearing surface, the engaging surface configured to engage the helical electrode. The engaging surface of the guide element insert interacts with the helical electrode to cause the helical electrode and the coupler to translate longitudinally when the helical electrode is rotated against the engaging surface, and longitudinal translation is limited by the distal end of the coupler contacting the proximal bearing surface of the guide element insert.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 is a combined cutaway and perspective view of an implantable medical device and lead in accordance with an embodiment of the present invention.

FIG. 2 is a side elevation view of the lead of FIG. 1.

FIG. 3A is a partial cross-sectional view of a lead in accordance with an embodiment of the present invention.

FIG. 3B is a partial cross-sectional view of the lead of FIG. 3A, shown in an extended position.

FIG. 4 is an end view of a distal assembly in accordance with an embodiment of the present invention.

FIG. 5 is a perspective view of the lead of FIG. 3.

FIG. 6 is a partial cross-sectional view of a lead in accordance with an embodiment of the present invention.

FIG. 7 is a perspective view of a guide element used in the lead of FIG. 6.

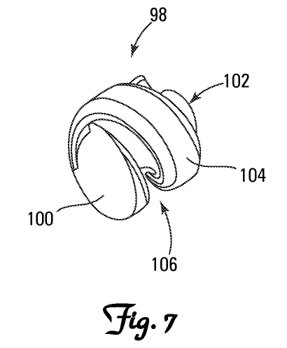

### DETAILED DESCRIPTION

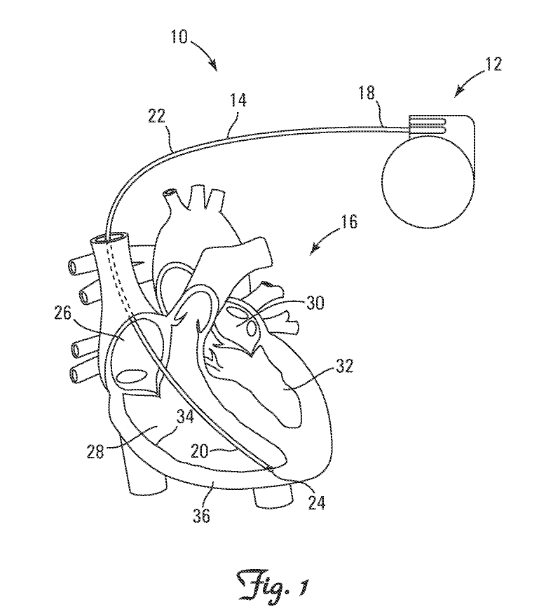

FIG. 1 is a perspective view of an implantable medical device (IMD) 10. The IMD 10 includes a pulse generator 12 and a cardiac lead 14. The lead 14 operates to convey electrical signals between the heart 16 and the pulse generator 12. The lead 14 has a proximal region 18 and a distal region 20. The lead 14 includes a lead body 22 extending from the proximal region 18 to the distal region 20. The proximal region 18 is coupled to the pulse generator 12 and the distal region 20 is coupled to the heart 16. The distal region 20 includes a fixation helix 24, which as will be discussed in greater detail with respect to subsequent drawings locates and/or secures the distal region 20 within the heart 16.

The lead body 22 can be made from any flexible, biocompatible materials suitable for lead construction. In various embodiments, the lead body 22 is made from a flexible, electrically insulative material. In one embodiment, the lead body 22 is made from silicone rubber. In another embodiment, the lead body 22 is made from polyurethane. In various embodiments, respective segments of the lead body 22 are made from different materials, so as to tailor the lead body characteristics to its intended clinical and operating environments. In various embodiments, the proximal and distal ends of the lead body 22 are made from different materials selected to provide desired functionalities.

As is known in the art, the heart 16 includes a right atrium 26, a right ventricle 28, a left atrium 30 and a left ventricle 32. It can be seen that the heart 16 includes an endothelial inner lining or endocardium 34 covering the myocardium 36. In some embodiments, as illustrated, the fixation helix 24, located at the distal region 20 of the lead, penetrates through the endocardium 34 and is imbedded within the myocardium 36.

In one embodiment, the IMD 10 includes a plurality of leads 14. For example, it may include a first lead 14 adapted to convey electrical signals between the pulse generator 12 and the right ventricle 28 and a second lead (not shown) adapted to convey electrical signals between the pulse generator 12 and the right atrium 26.

In the illustrated embodiment shown in FIG. 1, the fixation helix 24 penetrates the endocardium 34 of the right ventricle 28 and is embedded in the myocardium 36 of the heart 16. In some embodiments, the fixation helix 24 is electrically active and thus can be used to sense the electrical activity of the heart 16 or to apply a stimulating pulse to the right ventricle 28. In other embodiments, the fixation helix 24 is not electrically active. Rather, in some embodiments, other components of the lead 14 are electrically active.

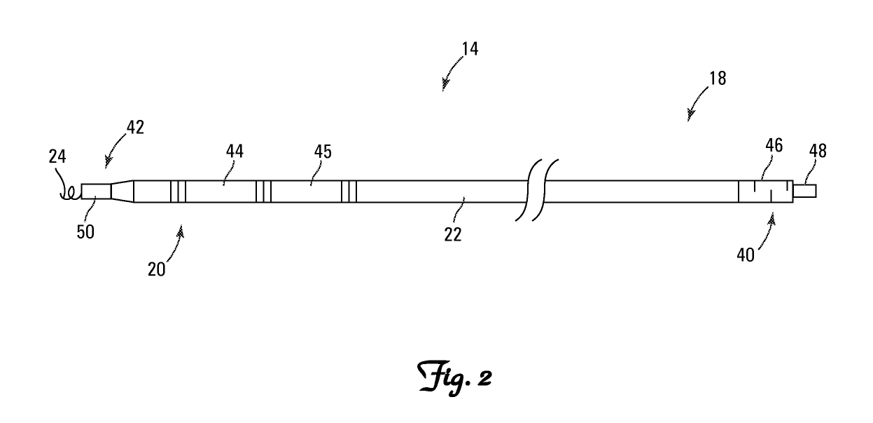

FIG. 2 is an isometric illustration of the lead 14. A connector assembly 40 is disposed at or near the proximal region 18 of the lead 14 while a distal assembly 42 is disposed at or near the distal region 20 of the lead 14. Depending on the functional requirements of the IMD 10 (see FIG. 1) and the therapeutic needs of a patient, the distal region 20 may include one or more electrodes. In the illustrated embodiment, the distal region 20 includes a pair of coil electrodes 44 and 45 that can function as shocking electrodes for providing a defibrillation shock to the heart 16. In some embodiments, coil electrodes 44 and 45 include a coating that is configured to control (i.e. promote or discourage tissue in-growth). In various embodiments, the lead 14 may include only a single coil electrode. In various other embodiments, the lead 14 includes one or more ring electrodes (not shown) along the lead body 22 in lieu of or in addition to the coil electrodes 44, 45. When present, the ring electrodes operate as relatively low voltage pace/sense electrodes. As will be appreciated by those skilled in the art, a wide range of electrode combinations may be incorporated into the lead 14 within the scope of the various embodiments of the present invention.

The connector assembly 40 includes a connector 46 and a terminal pin 48. The connector 46 is configured to be coupled to the lead body 22 and is configured to mechanically and electrically couple the lead 14 to a header on the IMD 10 (see FIG. 1). In various embodiments, the terminal pin 48 extends proximally from the connector 46 and in some embodiments is coupled to a conductor member (not visible in this view) that extends longitudinally through the lead body 22 such that rotating the terminal pin 48 (relative to the lead body 22) causes the conductor member to rotate within the lead body 22. In some embodiments, the terminal pin 48 includes an aperture extending therethrough in order to accommodate a guide wire or an insertion stylet.

The distal assembly 42 includes a housing 50, within which the fixation helix 24 is at least partially disposed. As will be explained in greater detail below, the housing 50 includes or accommodates a mechanism that enables the fixation helix 24 to move distally and proximally relative to the housing 50. In some embodiments, the distal assembly 42 may include structure (not seen in this view) that limits distal travel of the fixation helix 24 (relative to the housing 50) in order to reduce or prevent over-extension of the fixation helix 24. As noted above, the fixation helix 24 operates as an anchoring means for anchoring the distal region 20 of the lead 14 within the heart 16. In some embodiments, the fixation helix 24 is electrically active, and is also used as a pace/sense electrode. In some embodiments, the fixation helix 24 is made of an electrically conductive material such as Elgiloy, MP35N, tungsten, tantalum, iridium, platinum, titanium, palladium, stainless steel as well as alloys of any of these materials. In some embodiments, the fixation helix 24 is made of a non-electrically conductive material such as PES (polyethersulfone), polyurethane-based thermoplastics, ceramics, PEEK (polyetheretherketone) and polypropylene.

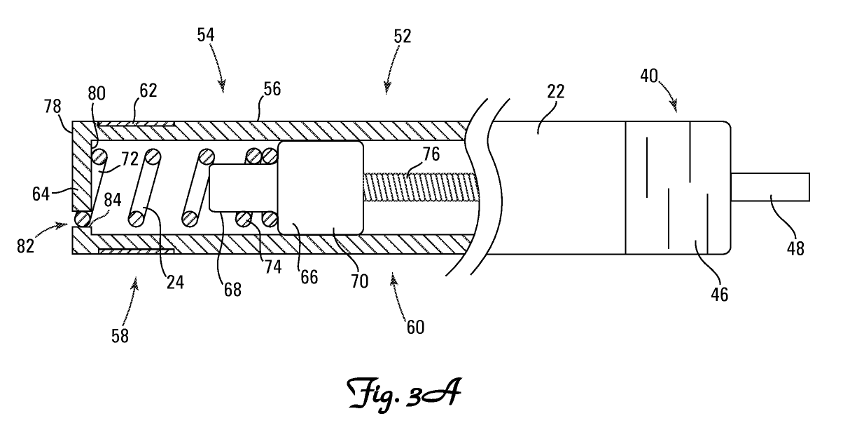

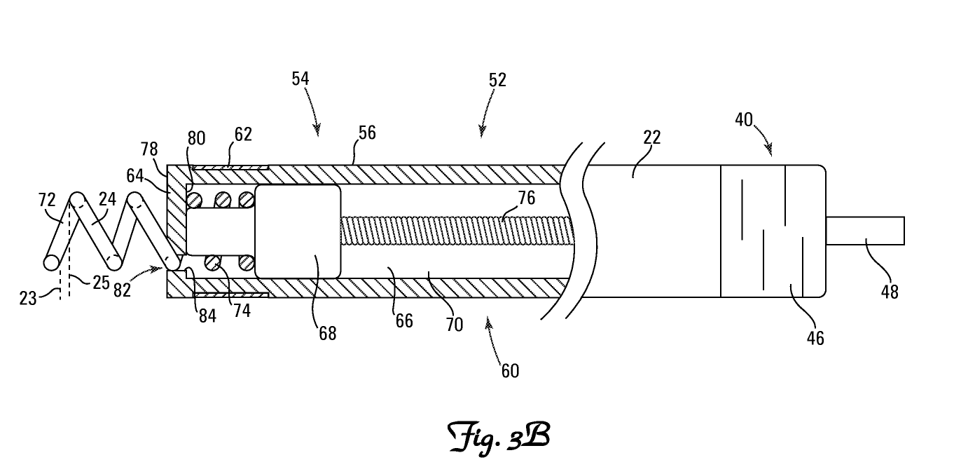

FIGS. 3-6 illustrate several embodiments of leads including distal assemblies in accordance with the present invention. FIGS. 3A and 3B are partial cross-sections of a lead 52 that includes a distal assembly 54. In FIG. 3A, the fixation helix 24 is illustrated in a retracted position while FIG. 3B illustrates the fixation helix 24 in an extended position. Other portions of the lead 52, such as the proximal region 18, are similar to corresponding portions of the lead 14 described above. In the illustrated embodiment, the fixation helix 24 is electrically active so as to be operable as a pace/sense electrode.

As shown in FIGS. 3A and 3B, the distal assembly 54 includes a housing 56 that includes a distal region 58 and a proximal region 60. The housing 56 is, in general, relatively rigid or semi-rigid. In some embodiments, the housing 56 is made of an electrically conductive material such as Elgiloy, MP35N, tungsten, tantalum, iridium, platinum, titanium, palladium, stainless steel as well as alloys of any of these materials. In some embodiments, the housing 56 is made of a non-electrically conductive material such as PES, polyurethane-based thermoplastics, ceramics, polypropylene and PEEK.

In the illustrated embodiment, a drug eluting collar 62 is disposed about an exterior of the housing 56 within the distal region 58. In various embodiments, the drug eluting collar 62 is configured to provide a time-released dosage of a steroid or other anti-inflammatory agent to the tissue to be stimulated, e.g., the heart tissue in which the electrically active fixation helix 24 is implanted. While not illustrated, in some embodiments the distal assembly 54 may include a radiopaque element disposed under the drug eluting collar 62.

The distal assembly 54 includes a guide element 64 that in some embodiments is integrally formed as part of the housing 56. In some embodiments, the guide element 64 is formed as a separate element that is subsequently attached or otherwise secured to the housing 56. While in the illustrated embodiment the guide element 64 is disposed at a distal end of the housing 56, in other embodiments the guide element 64 may be disposed somewhat proximal of the distal end of the housing 56.

A coupler 66 that has a distal portion 68 and a proximal portion 70 is disposed within the housing 56. The coupler 66 is formed of a metallic material and is configured to move longitudinally and/or rotationally with respect to the housing 56. In some embodiments, as illustrated, the distal portion 68 may have a relatively smaller diameter (relative to the proximal portion 70) in order to accommodate the fixation helix 24. While not illustrated, in some embodiments the proximal portion 70 is configured to accommodate a seal that provides a seal between the coupler 66 and the housing 56.

The fixation helix 24 has a distal region 72 and a proximal region 74. The proximal region 74 is secured to the distal portion 68 of the coupler 66. One or more attachment methods are used to secure the fixation helix 24 to the coupler 66. In some embodiments, the proximal region 74 of the fixation helix 24 is welded or soldered onto the distal portion 68 of the coupler 66. In some embodiments, the proximal region 74 of the fixation helix 24 has an inner diameter that is less than an outer diameter of the distal portion 68 of the coupler 66, and thus is held in place via compressive forces. In some embodiments multiple attachment methods are used.

A conductor member 76 is secured to the proximal portion 70 of the coupler 66, and extends proximally through the lead body 22 to the connector assembly 40. In some embodiments, the conductor member 76 includes or is otherwise formed from a metallic coil. The coupler 66 provides an electrical connection between the conductor member 76 and the fixation helix 24. In the connector assembly 40, the conductor member 76 is coupled to the terminal pin 48 such that rotation of the terminal pin 48 causes the conductor member 76 to rotate. As the conductor member 76 rotates, the coupler 66 and the fixation helix 24 will also rotate. The fixation helix 24 interacts with the guide element 64 such that rotation of the fixation helix 24 relative to the guide element 64 causes the fixation helix 24 to translate relative to the housing 56. In some embodiments, the fixation helix 24 is rotated via a stylet that is inserted through an aperture that may be formed within the terminal pin 48 (FIG. 2).

In the illustrated embodiment, the guide element 64 is formed as a distal plate. The guide element 64 includes an outer surface 78 and an inner or proximal bearing surface 80, as will be described in greater detail below. An opening 82 extends between the outer surface 78 and the proximal bearing surface 80. The opening 82 may be considered as being defined by a side wall that provides an engaging surface 84. As the fixation helix 24 rotates through the opening 82, individual turns of the fixation helix 24 interact with the engaging surface 84 in a threaded manner, causing the fixation helix 24 to move or translate in an axial direction relative to the housing 56. The engaging surface 84 both guides and engages the fixation helix 24.

A helical structure such as the fixation helix 24 can be considered as having a pitch, which can be defined as the axial distance between adjacent turns or windings, typically measured at a common radial position. In some embodiments, a half-pitch is a useful dimension. FIG. 3B includes a first dotted line 23 and a second dotted line 25. As shown, the first dotted line 23 can be seen as aligned with a back side of one turn of the fixation helix 24, while the second dotted line 25 can be seen as aligned with a front side of an adjacent turn of the helix 24. The distance between the back side of a turn and the front side of the proximally adjacent turn defines the half-pitch dimension of the helix 24. Thus, in FIG. 3B, the distance between the first dotted line 23 and the second dotted line 25 is the half-pitch of the fixation helix 24.

Relative dimensions of the fixation helix 24 and the guide element 64 may be selected to improve the interaction between the fixation helix 24 and the guide element 64. For example, the guide element 64 may have a thickness (between the outer surface 78 and the proximal bearing surface 80) that is determined, at least in part, as a function of the cross-sectional diameter of an individual turning of the fixation helix 24 and/or the pitch of the fixation helix 24 (i.e., the axial distance between adjacent turns of the fixation helix 24.

In some embodiments, the thickness of the guide element 64 may be determined to be in the range of about 50 percent to about 90 percent of the half-pitch defined above with respect to FIG. 3B. It will be appreciated that if the wall thickness (of the guide element 64) is too small relative to the cross-sectional diameter of an individual turning of the fixation helix 24 and/or the pitch of the fixation helix 24, the fixation helix 24 may bind against the guide element 64. If the wall thickness is too small, relatively speaking, there may be significant axial play between the fixation helix 24 and the guide element 64, which in some circumstances may be undesirable. It will be appreciated that changes in the pitch of the fixation helix 24 and/or the cross-sectional diameter of an individual turning of the fixation helix 24 may dictate a different wall thickness.

In some embodiments, the guide element 64 has a wall thickness (between the outer surface 78 and the proximal bearing surface 80) that is determined to guide the fixation helix 24 with minimal axial play without causing too much friction between the fixation helix 24 and the guide element 64. In a particular embodiment, the guide element 64 may have a wall thickness that is about 80 percent of the aforementioned half-pitch.

In some embodiments, the opening 82 has a width that is a function, at least in part, of the cross-sectional diameter of an individual turning of the fixation helix 24. In some embodiments, the opening 82 has a width that is about the same as or slightly larger than the aforementioned cross-sectional diameter. In some embodiments, these dimensional relationships improve the ease in which the fixation helix 24 may rotate through the guide element 64.

As the coupler 66 and the fixation helix 24 move distally relative to the housing 56, it can be seen that the distal portion 68 of the coupler 66 may contact the proximal bearing surface 80. Thus, in some embodiments, the proximal bearing surface 80 functions as a distal stop, limiting distal extension of the fixation helix 24 relative to the housing 56. In the illustrated embodiment, the distal portion 68 of the coupler 66 has a flat profile. In other embodiments, the distal portion 68 of the coupler 66 may have other shapes such as a spherical profile, a conical profile, and others.

In some embodiments, the distal portion 68 of the coupler 66 may include a distal extension that further limits relative distal travel of the coupler 66 as the distal extension would contact the proximal bearing surface 80 sooner. In some cases, the relative length of the coupler 66 may be varied to control how much axial movement of the coupler 66 (and the fixation helix 24) is permitted.

In some embodiments, such as that illustrated in FIG. 3A, the distal region 72 of the fixation helix 24 may extend at least partially into the opening 82 when the fixation helix 24 is at a proximal-most position. In some embodiments the lead 52 includes a proximal stop that limits relative proximal movement of the coupler 66 and thus the fixation helix 24.

In some embodiments, having the distal region 72 of the fixation helix 24 extend at least partially into the opening 82 provides advantages in locating the distal region 72 of the fixation helix relative to the opening 82. For example, configuring the distal assembly 54 such that the distal region 72 extends at least partially into the opening 82 when fully retracted relative to the housing 56 facilitates proper alignment and engagement between the fixation helix 24 and the opening 82.

FIG. 3B, in comparison, shows the coupler 66 and thus the fixation helix 24 in a distal-most position relative to the housing 56 (i.e., with the fixation helix 24 fully extended relative to the housing 56). In the illustrated position, the distal portion 68 of the coupler 66 can be seen as contacting the proximal bearing surface 80 of the guide element 64 while the distal region 72 of the fixation helix 24 extends distally from the housing 56.

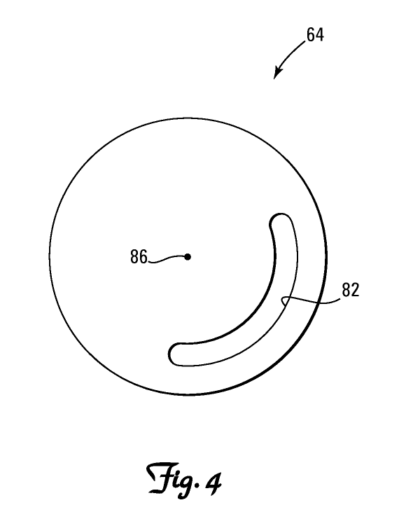

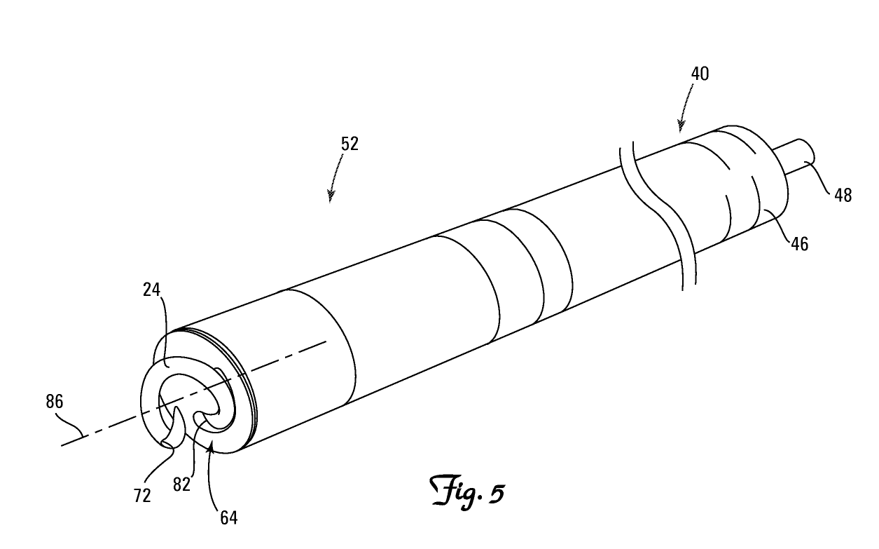

FIGS. 4 and 5 illustrate particular features of the guide element 64 in further detail. FIG. 4 is an end view with the fixation helix 24 removed for clarity while FIG. 5 is a perspective view in which the fixation helix 24 is seen extending through the guide element 64. In the illustrated embodiment, the opening 82 has an arcuate shape. In some embodiments, the opening 82 has a radius of curvature that substantially matches a radius of curvature of the fixation helix 24 and moreover is radially positioned relative to a center line 86 extending axially through the distal assembly 54 in order to align with the fixation helix 24. It can also be seen that the opening 82 has a radius of curvature that is radially positioned, relative to the center line 86, to at least substantially align with the radial position of the fixation helix 24. In some embodiments, the opening 82 has an inner curve that aligns with an inner diameter of the fixation helix 24 and an outer curve that aligns with an outer diameter of the fixation helix 24. In some embodiments, the opening 82 may have other shapes beyond the C-shaped opening that is illustrated.

In some embodiments, the structure and configuration of the guide element 64 may provide advantages. For example, the guide element 64 serves to guide and advance the fixation helix 24 as well as to provide the functionality of a distal stop in a single element and thus provides spatial advantages within the housing 56.

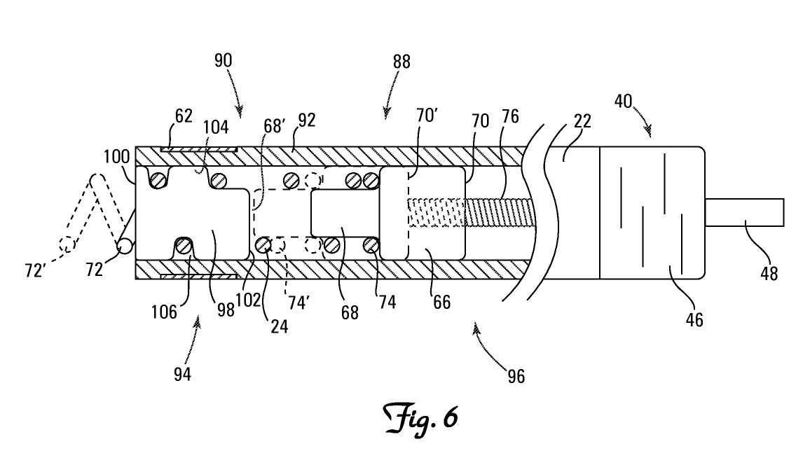

FIG. 6 is a partial cross-section of a lead 88 that includes an alternative distal assembly 90 according to another embodiment of the present invention. Except as explained in further detail below, in the embodiment of FIG. 6, elements that are substantially identical to corresponding elements of the lead 52 described above are given the same reference numbers as the corresponding elements in the lead 52. FIG. 6 illustrates a retracted or proximal-most position of the coupler 66 and the fixation helix 24 while the extended or distal-most position of the coupler 66 and the fixation helix 24 is shown in phantom. Moreover, hyphenated reference numbers are used to refer to the particular elements in the extended or distal-most position.

In the illustrated embodiment, the distal assembly 90 includes a housing 92 that includes a distal region 94 and a proximal region 96. The housing 90 can be formed of any of the materials discussed above with respect to the housing 56. In the illustrated embodiment, the drug eluting collar 62 may be disposed about an exterior of the housing 92 within the distal region 94. While not illustrated, in some embodiments the distal assembly 90 may include a radiopaque element disposed under the drug eluting collar 62.

The distal assembly 90 includes a guide element 98 having a distal surface 100 and a proximal bearing surface 102. In some embodiments, as illustrated, the guide element 98 is configured to be formed as a separate element and then be inserted into the housing 92. In some embodiments, the guide element 98 is dimensioned to have a compressive fit with the housing 92.

The housing 90 accommodates the coupler 66 and the fixation helix 24. As discussed with respect to FIGS. 3A and 3B, the proximal region 74 of the fixation helix 24 is attached to the distal portion 68 of the coupler 66. The conductor member 76 is secured to the proximal portion 70 of the coupler 66 and extends proximally to the lead body 22 to be secured to the connector assembly 40. In the connector assembly 40, the conductor member 76 is coupled to the terminal pin 48 such that rotation of the terminal pin 48 causes the conductor member 76 to rotate. As the conductor member 76 rotates, the coupler 66 and the fixation helix 24 will also rotate. The fixation helix 24 interacts with the guide element 98 such that rotation of the fixation helix 24 relative to the guide element 98 causes the fixation helix 24 to translate relative to the housing 92. In some embodiments, the fixation helix 24 is rotated via a stylet that is inserted through an aperture that may be formed within the terminal pin 48 (FIG. 2).

The guide element 98 has a distal surface 100 and a proximal surface that forms a proximal bearing surface 102. The guide element 98 also has an outer surface 104 that extends from the distal surface 100 to the proximal bearing surface 102 and that defines an engaging surface 106. In some embodiments, the distal surface 100 of the guide element 98 functions to limit material infiltration into an interior of the housing 90. In some embodiments, the proximal bearing surface 102 of the guide element 98 provides a surface against which the distal portion 68 of the coupler 66 may interact as the coupler 66 moves distally relative to the housing 92.

In the extended position, shown in phantom, it can be seen that the distal portion 68′ of the coupler 66′ has contacted the proximal bearing surface 102 of the guide element 98. The distal region 72′ of the fixation helix 24′ can be seen to have advanced distally from the housing 92 a distance sufficient for the fixation helix 24′ to penetrate the heart 16, as discussed previously with respect to FIG. 1.

In the illustrated embodiment, the engaging surface 106 forms a helical indentation having a profile that matches or at least substantially matches a profile of the fixation helix 24. As the fixation helix 24 rotates against the engaging surface 106, the fixation helix 24 and the engaging surface 106 interact in a threaded manner, causing the fixation helix 24 to move or translate in an axial direction. Thus, the engaging surface 106 both guides and engages the fixation helix 24. In some embodiments, the guide element insert 112 is formed of a polymeric material such as PEEK (polyetheretherketone) and is secured within the distal assembly 90 using adhesive or tack welding techniques.

In some embodiments, the structure and configuration of the guide element 98 may provide advantages. For example, the guide element 98 serves to guide and advance the fixation helix 24 as well as to provide the functionality of a distal stop in a single element. Moreover, the guide element 98 may provide manufacturing advantages as in some embodiments the remaining elements of the lead 88 may be assembled, then the guide element 98 may be threaded down over the fixation helix 24, into the interior of the housing 92, and then secured into position.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

We claim:

1. An implantable lead comprising:
a flexible body extending between a proximal end and a distal end;
a connector assembly secured to the proximal end for coupling the lead to an implantable medical device, the connector assembly including a terminal pin rotatable relative to the body;
a conductor member disposed longitudinally within the body and coupled to the terminal pin, the conductor member rotatable relative to the body; and
a distal assembly coupled to the distal end of the body and including:
a housing having a distal region and a proximal region, the proximal region fixedly coupled to the distal end of the body;
a coupler rotatably disposed within the housing, the coupler having a proximal end and a distal end, the proximal end connected to the conductor member;
a helical electrode fixedly secured to the coupler; and
a guide element connected to or integral with the housing located distal to the coupler, the guide element including an engaging surface and a proximal bearing surface, the engaging surface configured to engage the helical electrode;
wherein the engaging surface of the guide element interacts with the helical electrode to cause the helical electrode and the coupler to translate longitudinally when the helical electrode is rotated against the engaging surface, and wherein longitudinal translation of the coupler relative to the housing is limited by the distal end of the coupler contacting the proximal bearing surface of the guide element.

2. The implantable lead of claim 1, wherein the guide element comprises a distal plate disposed within the distal region of the housing, and an arcuate opening formed within the distal plate, the arcuate opening providing the engaging surface.

3. The implantable lead of claim 2, wherein the arcuate opening is configured to accommodate passage of the helical electrode therethrough.

4. The implantable lead of claim 2, wherein the arcuate opening is configured to threadedly engage the helical electrode.

5. The implantable lead of claim 2, wherein the distal plate comprises a rear surface that provides the proximal bearing surface.

6. The implantable lead of claim 1, wherein the guide element comprises an insert disposed within the distal region of the housing.

7. The implantable lead of claim 6, wherein the insert comprises a proximal end that provides the proximal bearing surface.

8. An implantable lead configured to carry an electrical signal, the implantable lead comprising:
a flexible body extending between a proximal end and a distal end, the body configured to carry an electrical signal from the proximal end to the distal end; and
a distal assembly coupled to the distal end of the body, the distal assembly including:
a housing having a distal region and a proximal region, the proximal region fixedly coupled to the distal end of the body, the distal region including a distal end;
a coupler rotatably disposed within the housing, the coupler having a proximal end and a distal end, the proximal end connected to the conductor member;
a fixation helix fixedly secured to the coupler; and
a guide element connected to or integral with the housing located distal to the coupler, the guide element including an engaging surface and a proximal bearing surface, the engaging surface configured to engage the helical electrode;
wherein the engaging surface of the guide element interacts with the fixation helix to cause the fixation helix and the coupler to translate longitudinally when the fixation helix is rotated against the engaging surface, and wherein longitudinal translation of the coupler relative to the housing is limited by the distal end of the coupler contacting the proximal bearing surface of the guide element.

9. The implantable lead of claim 8, wherein the guide element comprises a distal plate disposed within the distal region of the housing, an arcuate opening formed within the distal plate, the arcuate opening providing the engaging surface and a rear surface of the distal plate providing the proximal bearing surface.

10. The implantable lead of claim 9, wherein the distal plate is disposed at the distal end of the housing.

11. The implantable lead of claim 9, wherein the distal plate is disposed proximal to the distal end of the housing.

12. An implantable lead comprising:
a flexible body extending between a proximal end and a distal end;
a connector assembly secured to the proximal end for coupling the lead to an implantable medical device, the connector assembly including a terminal pin rotatable relative to the body;
a conductor member disposed longitudinally within the body and coupled to the terminal pin, the conductor member rotatable relative to the body; and
a distal assembly coupled to the distal end of the body and including:
a housing having a distal region, a distal end and a proximal region, the proximal region fixedly coupled to the distal end of the body;
a coupler rotatably disposed within the housing, the coupler having a proximal end and a distal end, the proximal end connected to the conductor member;
a helical electrode fixedly secured to the coupler; and
a distal plate connected to or integral with the housing located within the distal region of the housing, the distal plate including a proximal bearing surface and an arcuate opening defining an engaging surface, the engaging surface configured to engage the helical electrode;
wherein the engaging surface interacts with the helical electrode to cause the helical electrode and the coupler to translate longitudinally when the helical electrode is rotated against the engaging surface, and wherein longitudinal translation is limited by the distal end of the coupler contacting the proximal bearing surface of the distal plate.

13. The implantable lead of claim 12, wherein the helical electrode extends at least partially into the arcuate opening when the helical electrode is a proximal-most position fully retracted proximally relative to the housing.

14. The implantable lead of claim 12, wherein the arcuate opening has an aperture width that is about the same as a cross-sectional width taken through a single turning of the helical electrode.

15. The implantable lead of claim 12, wherein the fixation helix has a half-pitch, and the distal plate has a thickness that is about 50 to 90 percent of the half-pitch.

16. The implantable lead of claim 12, wherein the arcuate opening is positioned within the distal plate to radially align with the helical electrode.

17. The implantable lead of claim 12, wherein the arcuate opening has a radius of curvature that at least substantially matches a radius of curvature of the helical electrode.

18. An implantable lead comprising:
a flexible body extending between a proximal end and a distal end;
a connector assembly secured to the proximal end for coupling the lead to an implantable medical device, the connector assembly including a terminal pin rotatable relative to the body;
a conductor member disposed longitudinally within the body and coupled to the terminal pin, the conductor member rotatable relative to the body; and
a distal assembly coupled to the distal end of the body and including:
a housing having a distal region and a proximal region, the proximal region fixedly coupled to the distal end of the body;
a coupler rotatably disposed within the housing, the coupler having a proximal end and a distal end, the proximal end connected to the conductor member;
a helical electrode fixedly secured to the coupler; and
a guide element inserted within the distal region of the housing, the guide element including an engaging surface and a proximal bearing surface, the engaging surface configured to engage the helical electrode;
wherein the engaging surface of the guide element interacts with the helical electrode to cause the helical electrode and the coupler to translate longitudinally when the helical electrode is rotated against the engaging surface, and wherein longitudinal translation of the coupler relative to the housing is limited by the distal end of the coupler contacting the proximal bearing surface of the guide element.

19. The implantable lead of claim 18, wherein the engaging surface comprises a helical indentation that accommodates and guides the helical electrode in translating relative to the housing as the helical electrode rotates relative to the guide element.

20. The implantable lead of claim 18, wherein the helical indentation has a radius of curvature that at least substantially matches a radius of curvature of at least a portion of the helical electrode.

21. The implantable lead of claim 18, wherein the helical indentation has an axial distance between adjacent turns that at least substantially matches an axial distance between adjacent turns of the helical electrode.
